(19) United States
(12) Patent Application Publication
(10) Pub. No.: US 2026/0365955 A1
(43) Pub. Date: Nov. 19, 2026

(54) BIOLOGICAL MODELLING DEVICE

(71) Applicant: Aelius Biotech Limited, Newcastle upon Tyne Tyne and Wear (GB)

(72) Inventors: Peter Chater, Newcastle upon Tyne Tyne and Wear (GB); Matthew Wilcox, Newcastle upon Tyne Tyne and Wear (GB); Maria Zakhour, Newcastle upon Tyne Tyne and Wear (GB); Kyle Stanforth, Newcastle upon Tyne Tyne and Wear (GB); Jeffrey Pearson, Newcastle upon Tyne Tyne and Wear (GB); Anna-Marie Gorman, Durham (GB); Rhydian Lewis, Durham (GB); Craig Manning, Durham (GB)

(21) Appl. No.: 19/136,759

(22) PCT Filed: Dec. 6, 2023
(86) PCT No.: PCT/GB2023/053148

§ 371 Date: Jun. 6, 2025

(30) Foreign Application Priority Data
Dec. 7, 2022 (GB) 2218384.2

(51) Int. Cl. C12M 1/04; C12M 1/00; C12M 1/12; C12M 1/32; C12M 3/00

(52) U.S. Cl. CPC C12M 23/24; C12M 23/12; C12M 23/38; C12M 23/46; C12M 25/02; C12M 37/04

(57) ABSTRACT

The present invention provides a biological modelling device for the co-culture of cells, comprising: a first chamber providing a first reservoir, a second chamber providing a second reservoir, wherein the second reservoir and first reservoir are connected via a permeable support and the first chamber is at least partially received in the second chamber; and a seal arranged and sealingly engaging the first and second chambers such that a first gaseous environment of the first reservoir of the first chamber and a second gaseous environment of the second reservoir of the second chamber are separated by the seal.

The invention relates generally to a biological modelling device. In particular, the invention relates to a biological modelling device comprising two chambers, each with a gaseous environment, with an improved sealing arrangement for maintaining separation between the gaseous environments of the two chambers.

### BACKGROUND

Biological modelling devices are known to be used for in vitro cultivation of biological cells and/or tissue, in order to simulate in-vivo environments such as a luminal model of the digestive tract, for example. Typically, biological modelling devices have a plate with one or more indentations or ‘wells’ that are used to receive a biological element, such as a cell population or tissue sample, at an apical side. Some devices additionally have one or more cup-shaped inserts, each placed within a well. The inserts receive and hold a different biological element, such as a second cell population or tissue sample, at a basolateral side. Some known inserts have a membrane on a bottom surface. When nutrient media is introduced into one or more wells, and/or the insert, of the plate, the biological element(s) receive nutrients from the culture media.

It would be desirable to provide a biological modelling device with an improved sealing arrangement that maintains separation between the gaseous environment(s) of the two chambers. Particularly, it is an object of the invention to provide a biological modelling device providing sealingly separated gaseous environments between the wells and the inserts. It is an object of the invention to provide a biological modelling device in which the gaseous transfer pathway between the well(s) and the insert(s) is substantially through the adjoining membrane.

The present invention provides at least an alternative to biological modelling devices of the prior art.

### SUMMARY OF THE INVENTION

In accordance with the present invention there is provided a biological modelling device according to the appended claims.

According to an aspect of the present disclosure, there is provided a biological modelling device comprising two chambers,
a first chamber providing a first reservoir having a first gaseous environment, a second chamber providing a second reservoir having a second gaseous environment, wherein the second reservoir and first reservoir are connected via a permeable support and the first chamber is at least partially received in the second chamber; and
a seal arranged and sealingly engaging the first and second chambers such that the first gaseous environment of the first reservoir and a second gaseous environment of the second reservoir are separated by the seal.

In certain embodiments, the permeable support is a membrane.

In certain embodiments, the permeable support is an at least gas permeable membrane.

In certain embodiments, the permeable support is a porous surface allowing gas transfer between the first and the second reservoirs.

In certain embodiments, the permeable support is a porous surface allowing the passage of one or more of, at least: gas, nutrients and metabolites between the first and the second reservoirs.

In certain embodiments, the seal prevents the mixing of the first gaseous environment and the second gaseous environment other than through the permeable support. In this way, the device provides two separated gaseous atmospheres in the first and second chambers which are connected only through the permeable support for the purposes of gaseous and/or nutrient transfer between the two chambers. In this way the biological modelling device is provided with a sealing arrangement for maintaining sealed and distinct (i.e. separately controllable) gaseous environments in the first and second reservoirs. The configuration allows the introduction into, and sampling from, multiple chambers with distinct gaseous atmospheres without disturbing or mixing the gaseous atmospheres. Gas can transfer through the permeable support. There is diffusion/transfer of gas through the biological element supported by the permeable support, for example through the biological material/cells. By providing two gaseous environments sealed from one another except through the permeable support, the device is operable to biologically model conditions having an anaerobic and aerobic separation, and/or to test smoke, airborne toxins, drugs or the like across a biological element such as a colonic cell population, an epithelial cell population, an in vitro airway model or the like held on the permeable support.

When referred to herein, a “principal pathway” or a “primary pathway” is a pathway through which substantially all of the gaseous transfer occurs. In certain embodiments, the flow of gas between the first reservoir and the second reservoir is through the permeable support as a principal pathway. Any inherent gas permeability of the material of the seal is minor in comparison to the gas permeability of the permeable support.

In certain embodiments, the first gaseous environment comprises a headspace of the first reservoir of the first chamber.

In certain embodiments, the second gaseous environment comprises a headspace of the second reservoir of the second chamber.

When referred to herein, the “headspace” of a reservoir is the gaseous space above the biological element and/or any solution in the reservoir.

In certain embodiments the biological modelling device is useful for the co-culture of cells. In such a system, the first and the second chambers each comprise a biological element in the form of a cell(s) population and the principal pathway between the chambers is via the permeable support. In particular, a flow-path is provided through the permeable support as a principal (i.e. primary) pathway. One particular advantage of this arrangement, for example, is that an aerobic environment can be used to propagate one cell population, while an anaerobic environment can simultaneously be used to propagate another cell population. Each environment is therefore individually controllable, independent of other environments.

In certain embodiments, the second gaseous environment is sealed from the first gaseous environment by the seal and the first chamber in combination.

In certain embodiments, the first gaseous environment is an anaerobic environment.

In certain embodiments, the second gaseous environment is an aerobic environment.

In certain embodiments, the first and/or the second reservoir is configured to receive and retain at least one biological element. In certain embodiments the biological element may be one or more of: a mixed cell population, a single cell population, a co-culture of cells, a bacterial culture, a micro-organism population, a mucus or the like.

In addition, or alternatively, the first and/or the second reservoir is configured to receive and retain a solution. In certain embodiments the solution may be one or more of: a culture or nutritional medium, a buffering solution, a test solution or the like.

In certain embodiments, the seal is a gasket seal.

In certain embodiments, the permeable support is integrally formed with the gasket seal. In such embodiments, a separate first chamber element is not required and the gasket seal forms the first chamber.

In certain embodiments, the gasket seal comprises a pierceable portion.

In certain embodiments, the pierceable portion comprises a septum.

In certain embodiments, the septum is a resealable or self-sealing septum. In this way, a sampling or injection device can be introduced into the first or the second chamber independently of the other chamber. The gaseous environment of the chamber receiving the sampling or injection device remains sealingly separated from the other gaseous environment.

In certain embodiments, the first chamber is an insert comprising the permeable support.

In certain embodiments, the permeable support forms at least a portion of the bottom surface of the first chamber.

In certain embodiments, the insert comprises a wall upstanding from the permeable support bottom surface.

In certain embodiments, the first chamber is partially received through the gasket seal into the second chamber.

In certain embodiments, the seal comprises a barrier layer arranged to sealingly engage with the second chamber so as to separate the second gaseous environment from the first gaseous environment.

In certain embodiments, the barrier layer comprises an aperture.

In certain embodiments, the first chamber is partially received through the aperture to form a sealing engagement between the barrier layer and the first chamber whereby the second chamber is sealed by the barrier layer and the first chamber in combination.

In certain embodiments, said aperture of said barrier layer is a self-sealing septum. In this way, when the first chamber is pressed through the aperture, the barrier layer forms a sealing engagement, of the second chamber, with the first chamber.

In certain embodiments, the first chamber is a portion of the barrier layer.

In certain embodiments, the first chamber is integrally formed with the barrier layer.

In certain embodiments, the permeable support is integrally formed with the barrier layer. In such embodiments, a separate first chamber element is not required and the barrier layer forms the first chamber.

In certain embodiments, the second chamber is located in a base plate. In certain embodiments, the second chamber is formed integrally with the base plate.

In certain embodiments, the second chamber comprises a bottom surface, an inner wall extending upwardly from the bottom surface towards an open top, the bottom surface and inner wall together defining the second reservoir.

In certain embodiments, the biological modelling device comprises a plurality of second chambers, each second chamber comprising a second reservoir.

In certain embodiments, the biological modelling device comprises a plurality of first chambers, each first chamber corresponding to each of the plurality of second chambers, each first chamber comprising a first reservoir, and a permeable support located between the respective first and second reservoirs.

In certain embodiments, each first chamber is an insert.

In certain embodiments, each first chamber is formed integrally with the seal and wherein each first chamber is integrally formed with the permeable support.

In certain embodiments, the biological modelling device comprises a plurality of seals, each arranged between and sealingly engaging the respective first chamber and second chamber.

In certain embodiments, the seal is arranged between and sealingly engaging an outer surface of the first chamber and an inner wall of the second chamber.

In certain embodiments, the biological modelling device further comprises a cover for the, or each, first chamber, wherein the first chamber is closed by the cover.

In certain embodiments, the cover comprises a seal, arranged to sealingly engage the first chamber so as to separate the first gaseous environment from the surrounding ambient environment, and wherein the first chamber is sealed by the seal.

In certain embodiments, the seal of the cover comprises a pierceable portion.

In certain embodiments, the pierceable portion comprises a septum.

In certain embodiments, the septum is a resealable or self-sealing septum.

In certain embodiments, the seal of the cover comprises more than one, preferably two, pierceable portions.

In certain embodiments, one pierceable portion is configured to allow access to one of the first chamber or the second chamber, and another of said pierceable portions is configured to allow access to the other of said first chamber and the second chamber.

In certain embodiments, each pierceable portion comprises a septum.

In certain embodiments, the septum is a resealable or self-sealing septum.

In certain embodiments, the biological modelling device further comprises a transfer cover. More specifically, the transfer cover is formed of a gas permeable material. Yet more specifically, the transfer cover is configured and arranged to cover at least the, or each, of the first chamber(s). In this way, biological element(s) and/or solution(s) in the first chamber are prevented from transfer between adjacent first chambers.

In certain embodiments, the transfer cover is a gas permeable silicon membrane.

In certain embodiments, the first chamber is an apical chamber.

In certain embodiments, the second chamber is a basolateral chamber.

In certain embodiments, the first reservoir is configured to receive and hold a first culture media for feeding a first biological element. In certain embodiments, the second reservoir is configured to receive and hold a second culture media for feeding the first and/or the second biological element.

In certain embodiments, the second reservoir is configured to receive and hold a second culture media for maintaining the first biological element through the permeable support. More specifically, the second culture media may provide nutrients to the first biological element through the permeable support.

In certain embodiments, the first chamber comprises a seal plug operable to seal the first reservoir from the ambient environment. In this way, a first chamber may be closed and not used.

In some embodiments, the biological modelling device is a culture device for growing cells and/or tissue in vitro.

According to another aspect of the present disclosure, there is provided a method of growing cells and/or tissue in vitro using a biological modelling device according to another aspect of the invention.

According to a further aspect of the present disclosure, there is provided a biological modelling device, comprising:
a first chamber providing a first reservoir having a first gaseous environment, a second chamber having a second gaseous environment, wherein the second reservoir and first reservoir are connected via a permeable support and the first chamber is at least partially received in the second chamber; and
a seal arranged and sealingly engaging the first and second chambers such that a first gaseous environment of the first reservoir of the first chamber and a second gaseous environment of the second reservoir of the second chamber are separated by the seal; and
a cover for the first chamber, wherein the first chamber is closed by the cover, and wherein the cover comprises at least one aperture, and a duct extending from the at least one aperture towards the first chamber.

In certain embodiments, the cover further comprises at least one aperture and a duct extending from the at least one aperture towards the second chamber.

In certain embodiments, the first reservoir is configured to receive and hold a first biological element.

In certain embodiments, the second reservoir is configured to receive and hold a second biological element.

In certain embodiments, the first and/or the second biological element may comprise a cell population. More specifically, the cell population may be selected from one or more of the following: bacteria, epithelial cells, immune cells.

In certain embodiments the first and/or the second reservoir receive and hold a solution. More specifically, the solution may be one or more of: a culture or nutritional medium, a buffering solution, a test solution or the like.

According to another aspect of the present disclosure, there is provided a biological modelling device comprising:
a first chamber providing a first reservoir having a first gaseous environment, a second chamber providing a second reservoir having a second gaseous environment, wherein the second reservoir and first reservoir are connected via a permeable support and the first chamber is at least partially received in the second chamber; and
a seal arranged and sealingly engaging the first and second chambers such that a first gaseous environment of the first reservoir of the first chamber and a second gaseous environment of the second reservoir of the second chamber are separated by the seal; and
a cover for the first chamber, wherein the first chamber is closed by the cover, and wherein the cover comprises at least one aperture, and a duct extending from the at least one aperture towards the second chamber.

In certain embodiments, the cover further comprises at least one aperture and a duct extending from the at least one aperture towards the first chamber.

In certain embodiments, the first reservoir is configured to receive and hold a first biological element.

In certain embodiments, the second reservoir is configured to receive and hold a second biological element.

In certain embodiments, the first and/or the second biological element may comprise a cell population. More specifically, the cell population may be selected from one or more of the following: bacteria, epithelial cells, immune cells.

In certain embodiments the first and/or the second reservoir receive and hold a solution. More specifically, the solution may be one or more of: a culture or nutritional medium, a buffering solution, a test solution or the like.

### BRIEF DESCRIPTION OF THE DRAWINGS

Embodiments of the invention are now described, by way of example only, hereinafter with reference to the accompanying drawings, in which:

FIG. 1 illustrates an example of a biological modelling device, in: (a) an exploded perspective view; (b) an exploded perspective view from the front; (c) a perspective view of an assembled configuration; (d) a cross-sectional view from the front; and (e) a close-up cross-sectional view from the front;

FIG. 2 illustrates another example of a biological modelling device, in: (a) an exploded perspective view; (b) an exploded perspective view from the front; (c) a perspective view of an assembled configuration; (d) a cross-sectional view from the front; and (e) a close-up cross-sectional view from the front;

FIG. 3 illustrates the biological modelling device of FIG. 2, further including a sheet and pierceable film substrate, in (a) an exploded perspective view; (b) an exploded perspective view from the front; (c) a perspective view of an assembled configuration; (d) a cross-sectional view from the front; and (e) a close-up cross-sectional view from the front;

FIG. 4 illustrates another example of a biological modelling device, in (a) an exploded perspective view from the top; (b) an exploded perspective view from the bottom; and (c) a section view of an assembled configuration;

FIG. 5 illustrates a further example of a biological modelling device from a close-up view of the insert, in (a) an exploded perspective view; (b) a partial exploded perspective view; (c) a perspective view of an assembled configured; and (d) a section view of the assembled configuration;

FIG. 6 illustrates a portion of a base plate, in (a) a perspective view; and (b) a section view;

FIG. 7 illustrates a base plate of a biological modelling device printed using (a) fused deposition modelling (FDM); and (b) stereolithography (SLA);

FIG. 8 illustrates (a) a base plate of a biological modelling device in a perspective view; (b) an insert tray of the biological modelling device in a perspective view; (c) an assembly of the base plate and the insert tray in a perspective view; (d) an assembly of the base plate, insert tray and a cover in a perspective view; and (e) a cross section view of an assembly including the base plate and the insert tray;

FIG. 9 illustrates a model of a biological modelling device produced using stereolithography 3D printing, (a) with a separate base plate, insert tray and cover; (b) with an assembled base plate and insert tray, with a separate cover; and (c) an assembly of the base plate, insert tray and cover; and

FIG. 10 illustrates a biological modelling device comprising a transfer cover configured and arranged to prevent material transfer between adjacent first chambers.

### DETAILED DESCRIPTION

Certain terminology is used in the following description for convenience only and is not limiting. The words ‘right’, ‘left’, ‘lower’, ‘upper’, ‘front’, ‘rear’, ‘upward’, ‘down’ and ‘downward’ designate directions in the drawings to which reference is made and are with respect to the described component when assembled and mounted. The words ‘inner’, ‘inwardly’ and ‘outer’, ‘outwardly’ refer to directions toward and away from, respectively, a designated centreline or a geometric centre of an element being described (e.g. central axis), the particular meaning being readily apparent from the context of the description.

Further, as used herein, the terms ‘connected’, ‘attached’, ‘coupled’, ‘mounted’ are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, “first”, “second”, “third” etc. merely indicate that different instances of like objects are being referred to and are not intended to imply that the objects so described must be in a given sequence, either temporally, spatially, in ranking or in any other manner.

Like reference numerals are used to depict like features throughout.

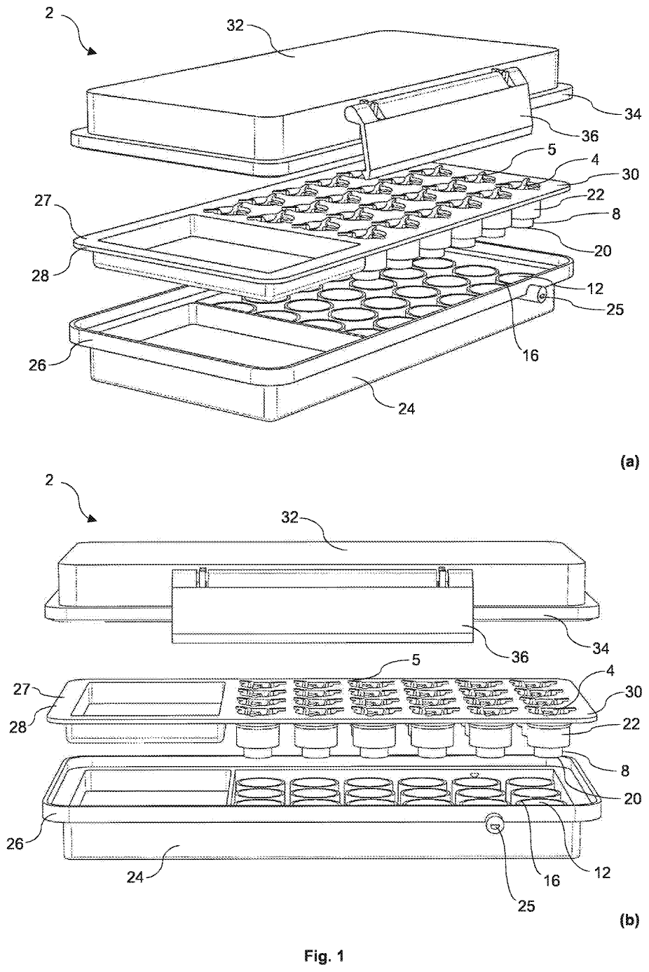

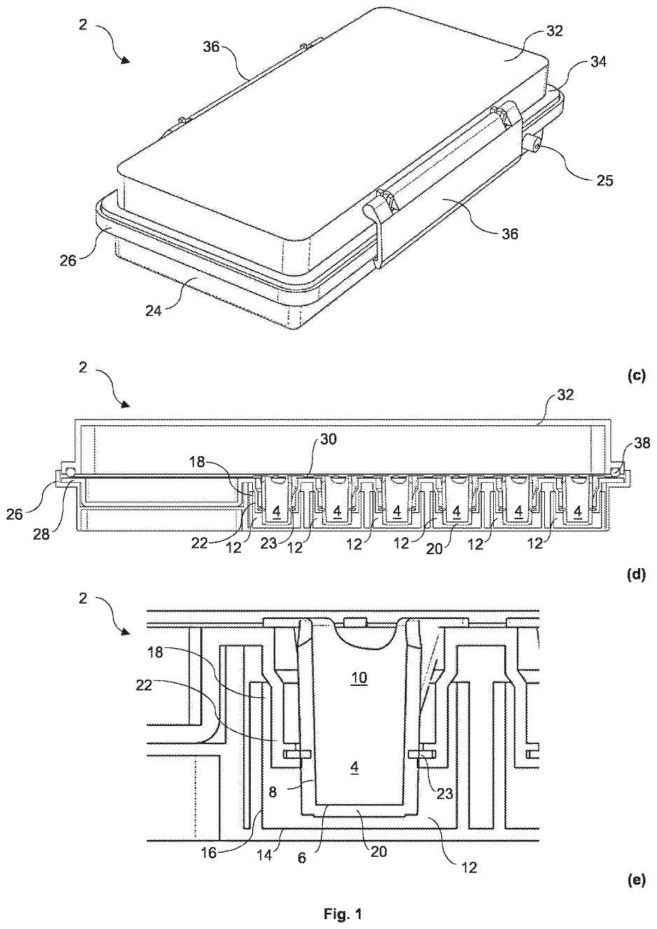

Referring now to FIG. 1, there is shown a biological modelling device 2. The biological modelling device 2 is provided with a number of inserts (i.e. first chambers) 4 which in this example, are inserted through apertures of a tray 27. In the depicted embodiments, the inserts 4 are provided with outwardly extending arms 5 that engage the surface of the tray 27 to hold the inserts 4 in place. Each one of the inserts 4 has an inner wall 8 with a tubular shape that terminates at a bottom surface 6 at one end and an open top at the other end. The tray 27 forms a gas tight seal around each insert 4. A porous membrane 20 is integrally formed with the bottom surface 6 of each insert 4. The inserts 4 define a reservoir for holding a biological material (e.g. cell population) to be cultured, such as a population comprising bacterial cells. In this example, the inserts 4 are apical chambers. The biological modelling device 2 is suitable for culturing cells and/or tissue in vitro. The pores of the membrane 20 are sized between 0.4 and 8 microns. In one particular example, to model the intestine, a membrane 20 having a pore size of 1.8 microns is used. The membrane 20 has a scaffold structure, which encourages tissue-like behaviour of the cultured cell. The scaffold structure may be a gel or other tissue culture matrix for example.

The device 2 is provided with wells (i.e. second chambers) 12 which are each formed as part of a base plate 24. The number of wells 12 corresponds to the number of inserts 4 in the tray 27. In this example there are provided twenty-four wells 12 that are arranged in a six-by-four matrix, but any number of wells 12 are envisaged depending on the requirements for cell propagation, such as six, twelve, twenty-four, forty-eight, ninety-six, three-hundred-and-eighty-four or one-thousand-five-hundred-and-thirty-six wells 12, for example. Each well 12 has an inner wall 16 with a tubular shape that terminates at a bottom surface 14 at one end and an open top at the other end. The inserts 4 are arranged in the tray 27 to align with a corresponding well 12 of the base plate 24 such that a central axis of each insert 4 and a central axis of each well 12 is aligned. The inserts 4 have a diameter that is smaller than that of the corresponding tubular shaped well 12. The wells 12 each define a reservoir for holding a biological material, such as a population comprising epithelial cells. The inserts 4 and the wells 12 in this example are tubular in shape, but other regular and irregular shapes are also envisaged. In this example, the wells 12 are basolateral chambers.

A seal 22 is provided surrounding the, or each, insert 4. In this example, separate seals 22 are provided which surround each of the inserts 4. The seals 22 are sized such that the inserts 4 of the tray 27 are received through the seals 22 and placed into the wells 12 of the base plate 24. When an insert 4 is received in the respective well 12 in this way, the seal 22 engages with the insert 4 and the well 12, so as to provide a sealing arrangement that separates a gaseous environment within the headspace of the insert 4 from a gaseous environment within the headspace of the well 12. In particular, the seal 22 is arranged between an outer surface of the insert 4 and the inner wall 16 of the well 12. Sealing is provided by the headspace 18 of the well 12 being closed by the seal 22 and the insert 4. In this particular example, a gasket 23 is provided to improve sealing between the insert 4 and the well 12, as best seen in FIG. 1(e).

The base plate 24 is provided with a skirt 26 that extends continuously around the periphery of the base plate 24. The tray 27 is provided with a flange 28 that extends outwardly of the tray 27. The tray 27 is sized and shaped such that the flange 28 of the tray 27 is retained within the base plate 24 by the skirt 26 of the base plate 24. When the tray 27 is held within the base plate 24 in this way, each insert 4 is aligned with and inserted towards and into the respective well 12. At the same time, the seal 22 contacts the inner wall 16 of the well 12 to provide a sealed environment within the reservoir of the well 12. The seals 22 are formed together with a barrier layer 30 that cooperates with the inserts 4, sealing the wells 12. One surface of the base plate 24 is provided with an air valve 25 that provides a free flow of air from the surrounding ambient environment outside the device 2 and into the base plate 24 and, more specifically, into the wells 12. This maintains an aerobic environment in the wells 12. It is envisaged that the air valve 25 may be provided with a particulate filter.

The reservoirs of the, or each insert 4, are connected to the respective well 12 through the porous membrane 20. The porous membrane 20 is integrally formed with the bottom surface 6 in this example, but it is envisaged that the porous membrane 20 may be provided as a separate component placed onto the bottom surface 6 of the insert 4. The porous membrane 20 may be provided in the form of a mesh, or a matrix of pores. The porosity of the membrane 20 allows for the passage of, for example, gas, nutrients and metabolites between the respective reservoirs of the insert 4 and the well 12.

The biological modelling device 2 is provided with a cover 32 that, when in the closed position, encloses the headspace 10 of the insert 4. A lower part of the cover 32 is provided with a flange 34 that extends around the periphery of the cover 32. A seal 38 is arranged in engagement with the flange so that the cover 32 is sealed against the tray 27 and the base plate 24, when the cover 32 is shut. The seal 38 may be a gasket seal. Opposite outward facing surfaces of the cover 32 each have a latch 36 attached via a hinge to the surface of the cover 32 which enables each latch 36 to independently pivot about its hinge with respect to the cover 32.

In use, with reference to FIG. 1(c) to (e), the inserts 4 are each provided with a first biological element (e.g. bacterial cells) and a maintenance medium, such as phosphate-buffered saline (PBS). The wells 12 are each provided with a second biological element (e.g. epithelial cells) and a different culture medium for culturing the cells. The tray 27 is lowered into the base plate 24 such that the inserts 4 are each lowered into and received by the respective well 12, with the porous membrane 20 interposing the reservoirs of the inserts 4 and wells 12. When the tray 27 is fully received within the base plate 24, the flange 28 of the tray 27 is retained by the skirt 26 of the base plate 24. In this arrangement, the seal 22 engages with the inner wall 16 of the well 12 and with the insert 4, such that the headspace 18 of the well 12 is closed by the seal 22 and the insert 4. As such, the gaseous environment in the headspace 10 of the insert 4 and the gaseous environment in the headspace 18 of the well 12 are separated by the seal 22. At the same time, the porous membrane 20 provides a path for gas, nutrients and/or metabolites to transfer between the reservoirs of the respective inserts 4 and wells 12.

When the tray 27 and base plate 24 are assembled together to receive the inserts 4 in the wells 12, the headspace 10 of each insert 4 is open to the surrounding environment. To close off the headspace 10 of the inserts 4 from the environment, the cover 32 is placed over the tray 27 and the latches 36 are pivoted about their hinge to engage with a bottom surface of the skirt 26.

The lower chamber (i.e. well 12) may be referred to as a basolateral chamber, and the upper chamber (i.e. insert 4) may be referred to as an apical chamber. In use, the upper chamber may comprise bacterial cells which are cultured in an anaerobic environment. A sachet may be provided in the chamber to create an anaerobic environment. The contents of the sachet initiates a reaction that quenches the oxygen in the environment. The lower chamber may comprise mammal epithelial cells which are cultured in an aerobic environment. In this example the aerobic environment may be provided by the air valve 25 that allows the free flow of air into the lower chamber. However, it is envisaged in other examples, both the upper and lower chambers could be fed from an external gas supply. Thus, the gaseous environment of the upper chamber is separated from the gaseous environment of the lower chamber, to provide a controlled way of co-culturing cells each under separate gaseous environments. The upper chamber may be used to model the lumen. The lower chamber may be used to model the intestinal wall. It is envisaged that a mucus layer may be additionally formed on the porous membrane 20. In particular, it is envisaged that a mucus layer may be formed on top of the epithelial cells to simulate intestinal epithelia. It is also envisaged that if some inserts 4 are not used, the open top of the insert 4 may be sealed by a seal plug (not shown). In this way, redundant inserts 4 are closed and not used.

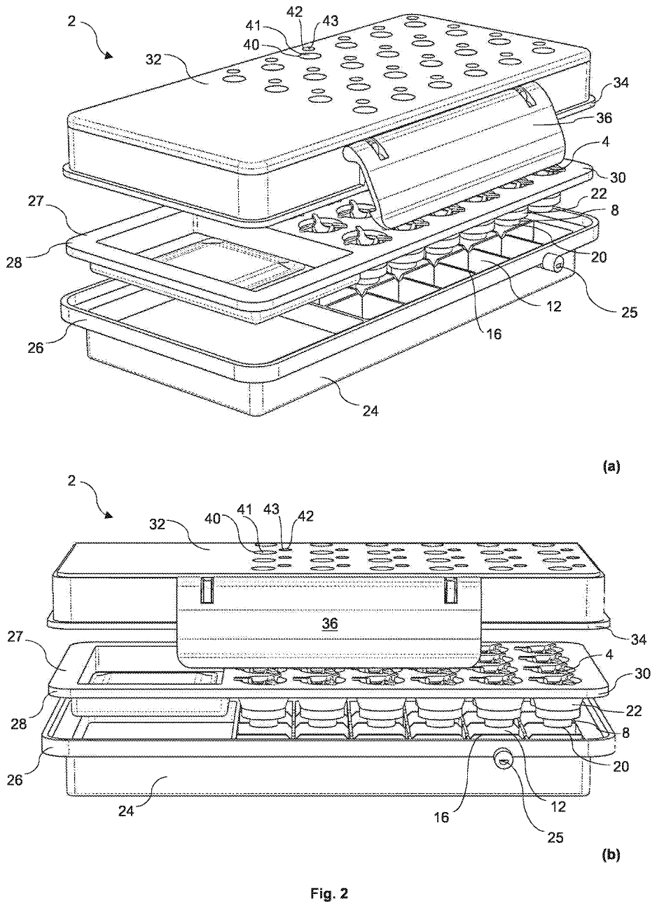

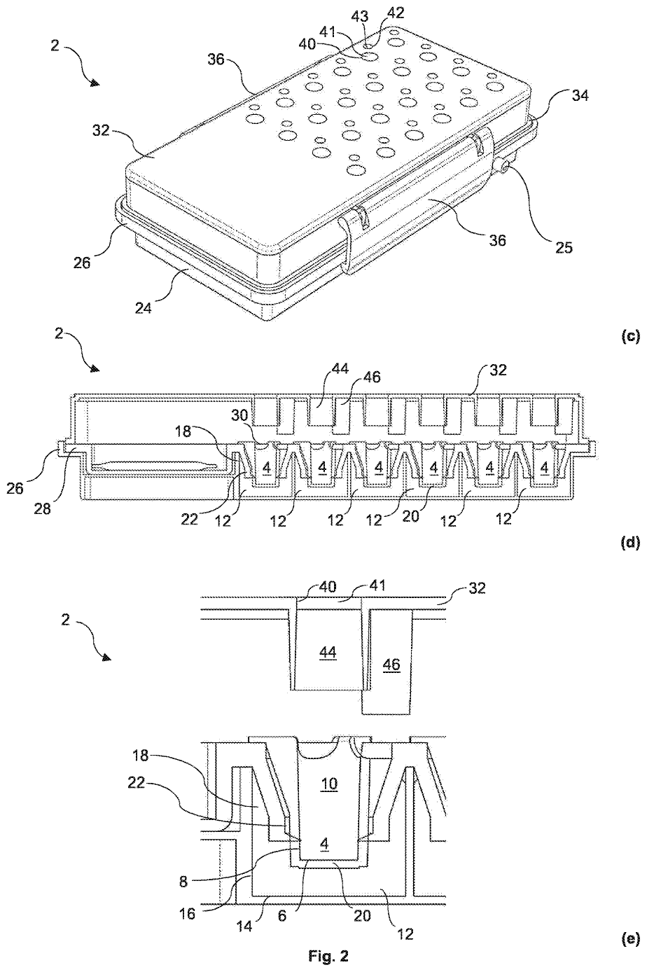

FIG. 2 shows another example of a biological modelling device 2, in which the wells 12, the seals 22 and the cover 32 are different from those in the example shown in FIG. 1. The biological modelling device 2 is provided with a plurality of wells 12 formed as part of a base plate 24. Each well 12 in this embodiment has an inner wall 16, with a square or rectangular footprint, terminating at a bottom surface 14 and an open top opposite the bottom surface 14. The wells 12 each define a reservoir for holding a biological material such as epithelial cells. The biological modelling device 2 is provided with inserts 4 in the same manner as in FIG. 1, and will therefore not be described again in detail here.

A seal 22 is provided surrounding the, or each, insert 4 such that the inserts 4 are received through the seal 22 and placed into the wells 12 of the base plate 24. The seals 22 in this example have a frustoconical outer profile that tapers inwards towards the base plate 24. In use, when the tray 27 is received on the base plate 24, the inserts 4 are lowered into the respective wells 12. When the inserts 4 are lowered into the wells 12 in this way, the lower end of the seals 22 engage with inner walls 16 of the wells 12, such that the headspace 18 of the wells 12 are closed and sealed by the seals 22 and the respective inserts 4. It is envisaged that the seals 22 may have a pierceable portion. The pierceable portions may have a resealable septum. In specific examples, it is envisaged that the pierceable portions of the seals 22 may be self-sealing septa.

The biological modelling device 2 is provided with a cover 32 that, when in the closed position, encloses the headspace 10 of the inserts 4. The cover 32 in this example is different from the embodiment in FIG. 1, in that there is provided an array of first apertures 40 and second apertures 42. The diameter of the first apertures 40 is larger than the diameter of the second apertures 42. The first apertures 40 are arranged in a six-by-four matrix which are aligned with a central axis of the respective inserts 4. The second apertures 42 are each arranged offset from the first apertures 40, and therefore also offset from the central axis of the respective inserts 4. Each of the first apertures 40 are covered by a pierceable film 41. Similarly, each of the second apertures 42 are covered by a pierceable film 43. The pierceable films 41,43 have a resealable septum. More specifically, the pierceable films 41,43 have a self-sealing septum.

As shown in FIG. 2(d) and 2(e), the first apertures 40 each lead to a duct 44 that provides a passageway directed towards the respective aligned insert 4, for introducing/extracting media and/or cells from the insert 4 when the film 41 is pierced. In this example, the second apertures 42 each lead to a separate duct 46 that provides a passageway directed towards the respective well 12, for introducing/extracting media and/or cells from the well 12 when the film 43 is pierced. Although in this example, it is shown that the ducts 44,46 extend linearly downwards, in other examples it is envisaged that the ducts 44,46 may instead extend in a different direction towards the inserts 4 and/or wells 12, or in a non-linear manner. For example, the ducts 44,46 may extend at an angle offset from the central axis of the wells 12 by 20 degrees. In an arrangement, not shown, ducts 44, 46 extend into the insert 4 and act as a condensation collector. In this way, condensation can be collected whilst preventing biological material, for example bacteria, transferring between adjacent inserts 4. Thus, the sterility of the device may be improved. The gas permeability of the ducts 44, 46 ensures a continuous first gaseous environment between adjacent upper chambers (e.g. inserts 4).

In this example, each of the wells 12, the seals 22 and the cover 32 are modified from the example in FIG. 1, but other examples are also envisaged in which one or more, but not all, of the wells 12, seals 22, and cover 32 are modified from the example in FIG. 1. For example, it is envisaged that a biological modelling device 2 may include all of the features of FIG. 1, wherein the cover 32 is modified to include the apertures 40,42 and the respective pierceable films 41,43. A separate example of a biological modelling device 2 may include a cover 32 with the apertures 40,42, the pierceable films 41,43, and the ducts 44,46.

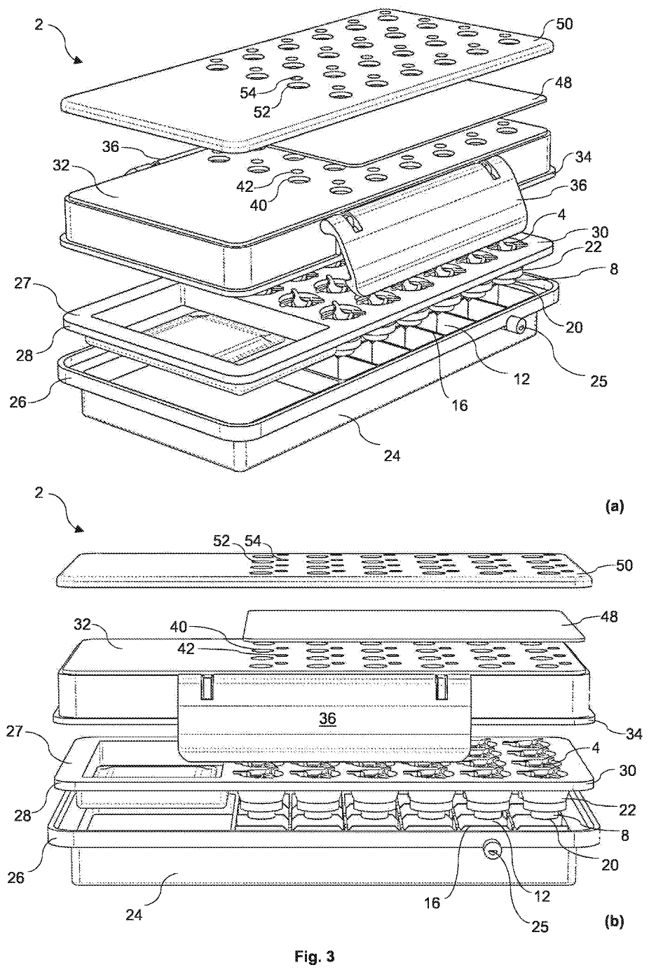

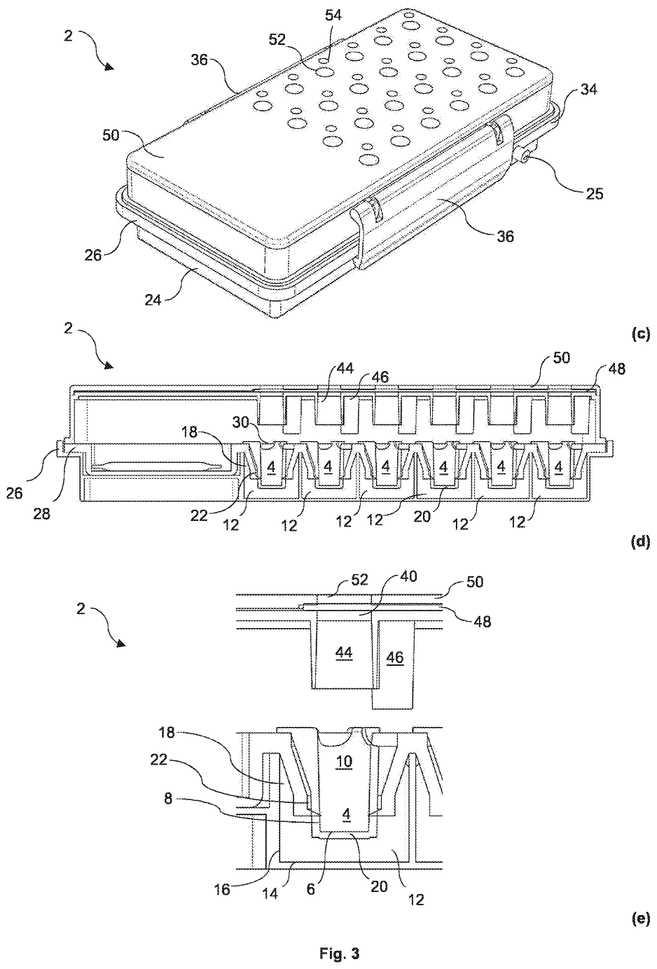

FIG. 3 shows a further example of a biological modelling device 2 that is substantially the same as the device 2 in FIG. 2. However, the larger first apertures 40 are not sealed by a pierceable film 41. Similarly, the smaller second apertures 42 are not sealed by a pierceable film 43. Rather, the apertures 40,42 are through-holes extending the entire thickness of the cover 32. The biological modelling device 2 in FIG. 3 is provided with a sheet 50 having apertures 52,54 that are arranged to align with the larger first apertures 40 and the smaller second apertures 42 in the cover 32 respectively. In particular, the apertures 52 of the sheet 50 are correspondingly sized and spaced to align with the larger first apertures 40 of the cover 32. The apertures 54 of the sheet 50 are correspondingly sized and spaced to align with the smaller second apertures 42 of the cover 32. A pierceable substrate layer 48 is arranged between the cover 32 and the sheet 50. The pierceable substrate layer 48 is sized and arranged so that it covers and extends between the apertures 40,42 of the cover 32 and the apertures 52,54 of the sheet 50.

In this example, the pierceable substrate layer 48 has a resealable septum. It is envisaged that the pierceable substrate layer 48 may have a self-sealing septum. In use, the substrate layer 48 is pierced through the aperture 52 to access an insert 4 below through the duct 44, to introduce/extract media and/or cells from the insert 4. The substrate layer 48 is pierced through the aperture 54 to access a well 12 below through the duct 46, to introduce/extract media and/or cells from the well 12.

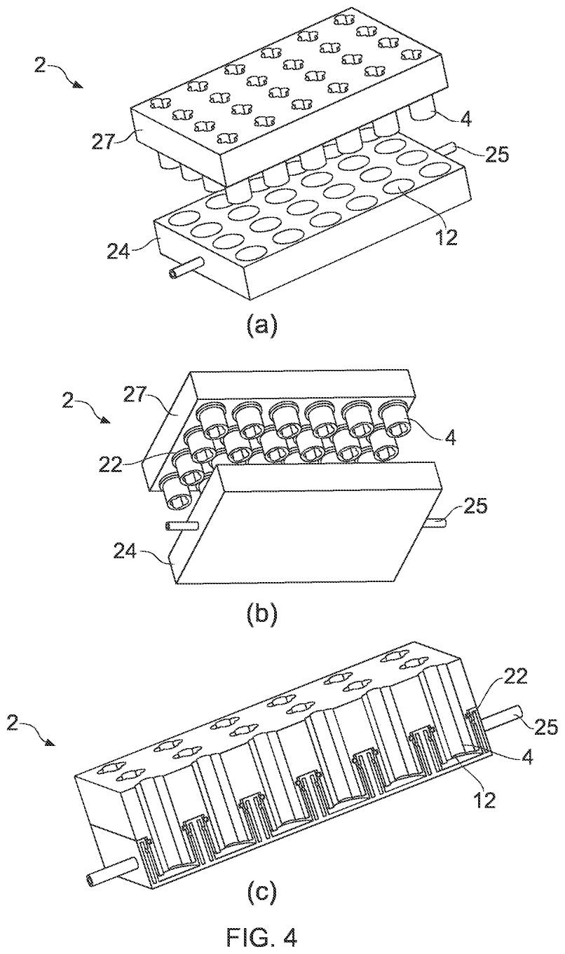

FIG. 4 shows an embodiment of a biological modelling device 2 with an alternative sealing arrangement to the previous examples. Each insert 4 of the tray 27 is provided with a seal in the form of an O-ring 22 that is received around the insert 4 and in abutment with the lower surface of the tray 27. When the inserts 4 are received within the corresponding wells 12 of the base plate 24, the chambers of the wells 12 are separated from the chambers of the inserts 4. The O-rings 22 provide a fluid-tight and gas-tight seal between the inserts 4 and the wells 12. The base plate 25 is provided with an air-valve-25 that can be connected to external tubes or pipes for introducing a gas or fluid into the wells 12. Since the O-rings 22 are provided above the chambers of the wells 12, gas is able to pass through the entirety of the base plate 24 and inside the wells 12 during use. In this example, the O-ring 22 is made from nitrile rubber, with an internal diameter of 14 mm, and an outer diameter of 18 mm.

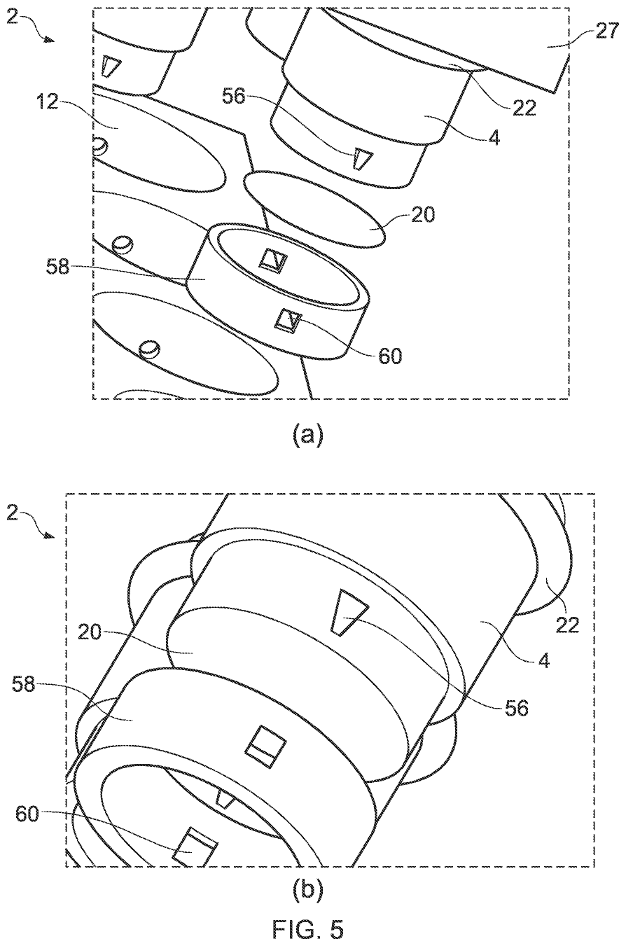

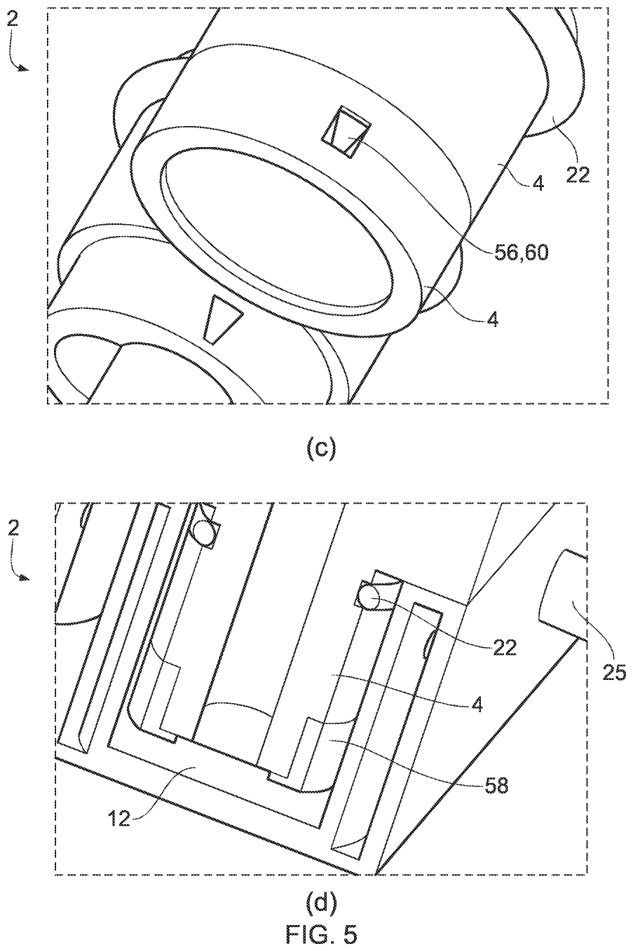

FIG. 5 shows a further modification to the biological modelling device 2 in which a porous membrane 20 is attached onto the bottom of the insert 4 and held in place by a clip 58. The insert 4 is provided with a protrusion 56 on opposite sides on a lower surface. The protrusions 56 engage with opposing passages (i.e. cut-outs) 60 of the clip, which hold the clip in place around the outer wall of the insert 4. The porous membrane 20 is sized to be larger than the opening of the clip 58, such that the porous membrane 20 urges against the insert 4 and retains it in position. By providing a clip 58 in this manner, an adhesive is not required to attach the porous membrane 20 in place. In this example, the gap between the bottom of the clip 58 and the bottom of the well 12 is two millimetres (mm).

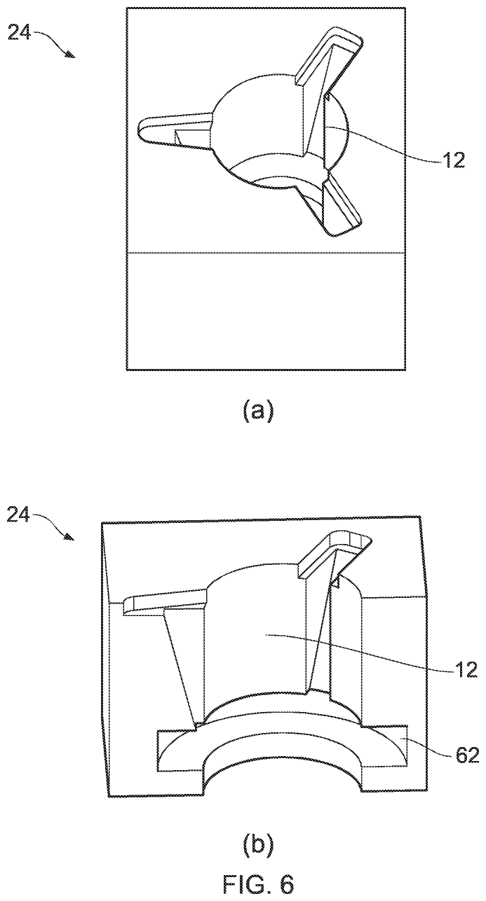

FIG. 6 shows an alternative embodiment showing a portion of a base plate 24 with a single well 12. The well 12 is provided with a recess (i.e. slot) 62 at a distal end that receives an O-ring seal. By providing an O-ring within the recess 62, a seal is formed between the base plate 24 and an insert when received in the well 12.

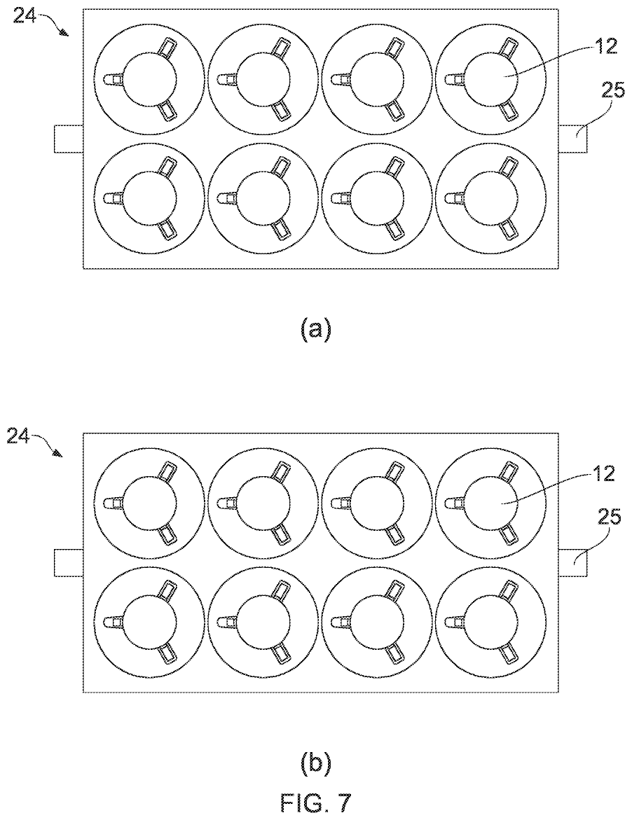

FIG. 7 shows two different samples of a base plate 24 that are produced using different 3D printing techniques. FIG. 7(a) shows a base plate 24 that is 3D-printed using fused deposition modelling (FDM), with acrylonitrile butadiene styrene (ABS) as a printing material. FIG. 7(b) shows a base plate 24 that is 3D-printed using stereolithography (SLA), with Biomed clear resin as a printing material.

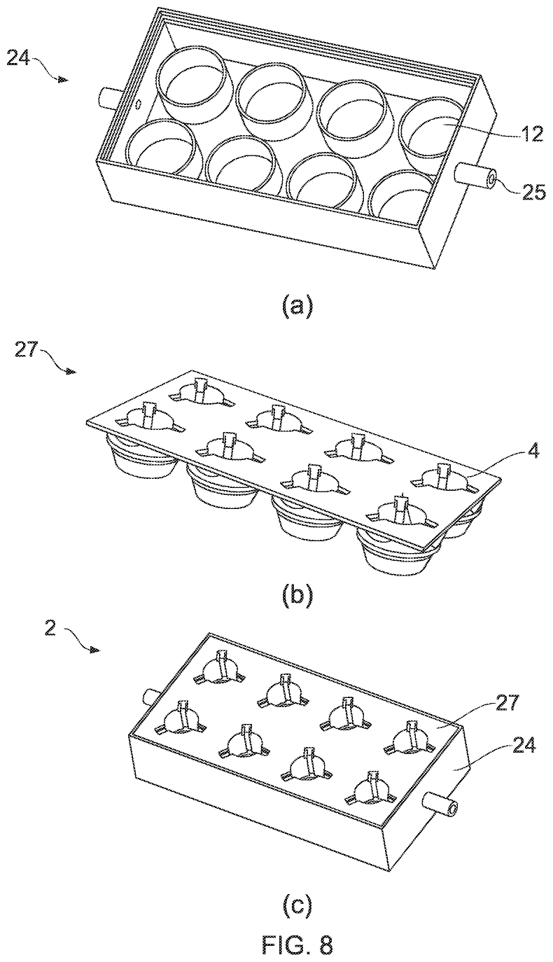

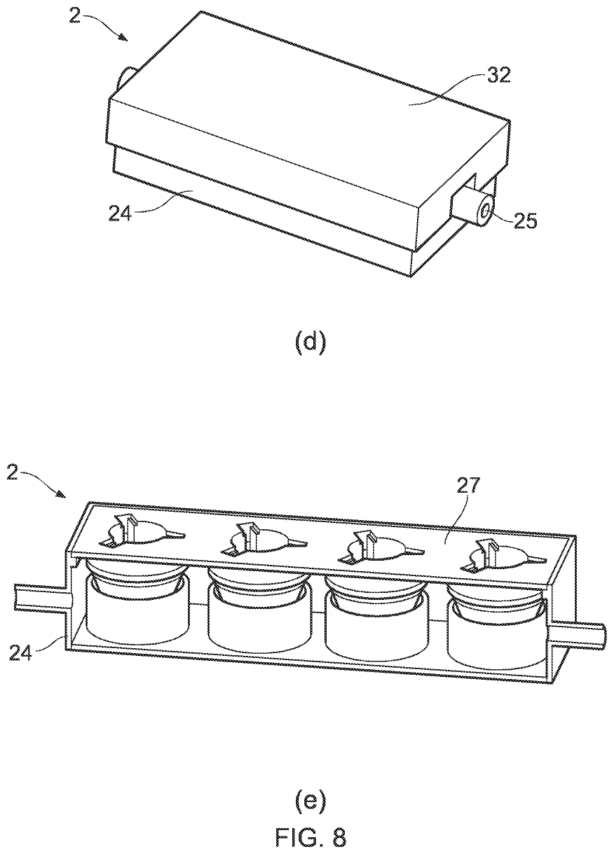

FIG. 8 shows a various components of a biological modelling device. FIG. 8(a) shows a base plate 24 having eight tubular-shaped wells 12, and valves 25 at either end to introduce a gas or fluid into the wells 12 during cell culture. FIG. 8(b) shows a tray 27 including eight inserts 8 that are shaped and sized to be held within the wells 12. The base plate 24 and the tray 27 are assembled together in use, and optionally, a cover 32 is provided to prevent evaporation of the contents within the device, as shown in FIG. 8(d).

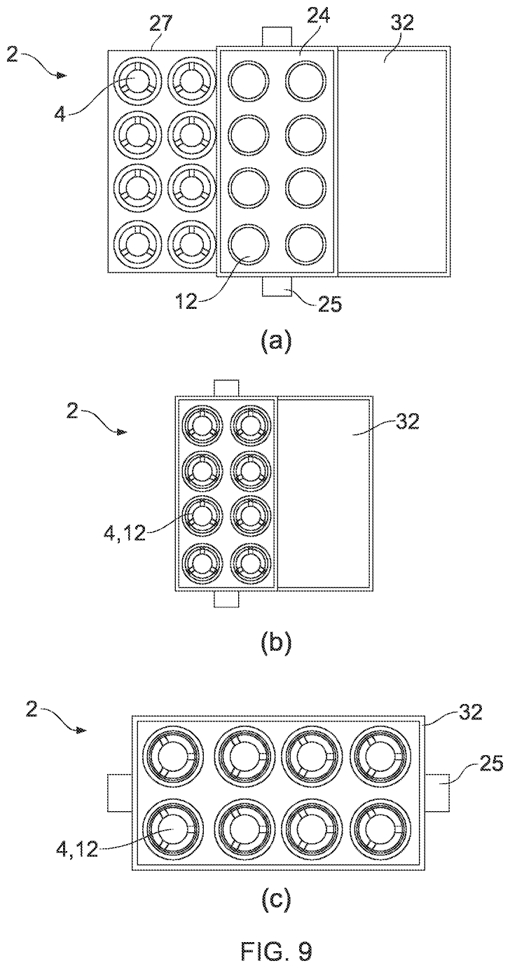

As shown in FIG. 9, each of the base plate 24, the insert tray 27, and the cover 32 may be separately manufactured (e.g. by 3D printing). In this particular example, the components are printing using stereolithography 3D printing using acrylonitrile butadiene styrene (ABS) as a printing material. To assemble the biological modelling device 2, the tray 27 is placed onto the base plate 24 such that the inserts 4 are held within the corresponding wells 12. The cover 32 is then placed on top of the tray 27 and base plate 24 assembly, to prevent the contents inside from evaporating during cell culture. The base plate 24 is provided with a valve 25 which may be attached to external pipes or tubing (not shown) to introduce a fluid or gas into the chambers of the wells 12. For example, oxygen may be introduced through the valve 25 to create an aerobic environment in the wells 12.

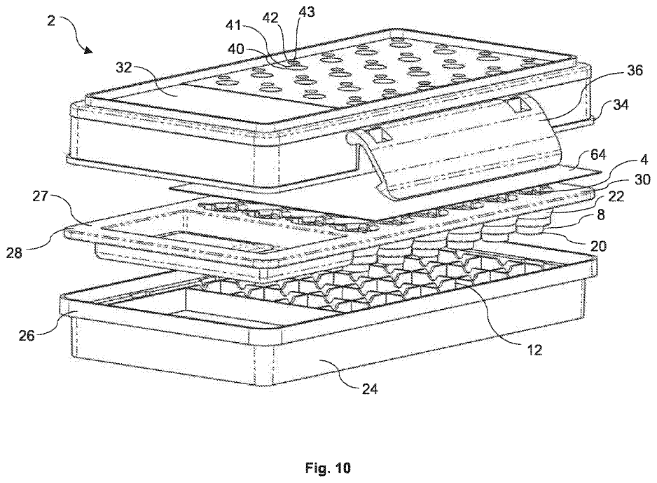

FIG. 10 illustrates the biological modelling device 2 with a gas permeable silicon transfer cover 64. The transfer cover 64 lies on top of insert tray 27 and prevents biological material and/or solution transfer occurring between adjacent inserts 4. The gas permeability of the transfer cover 64 ensures a continuous first gaseous environment between adjacent upper chambers (e.g. inserts 4).

The biological modelling device of the present invention is suitable for use in a number of exemplary models. The following models and biological materials are provided by way of example.

### Human Colonic Model

A representative colonic cell line (such as CACO-2 or T84) is grown in a monolayer on a transwell insert 4. A cell compatible mucus layer is placed on top of the cell layer in the first chamber provided by the insert 4. A bacterial inoculum and nutritional media is placed on top of mucus layer. The bacterial inoculum may be one of: single bacteria, multiple bacteria, or a representative microbial culture grown from faecal inoculum.

The insert 4 provides an upper chamber having an anaerobic environment (less than about 0.5-1% oxygen), generated by GasPak™. The lower chamber comprises wells 12 in which there is provided an aerobic environment. The lower chamber may hold a nutritional media and, optionally, a mixed cell population including immune cells and/or fibroblasts and/or epithelial cells and/or blood cells.

Samples may be taken from either chamber (insert 4 and/or wells 12) throughout a modelling regime or at the end or at a specified time point. Samples may be analysed for metabolites, material transfer, 16s sequencing of bacteria, RNA sequencing from cells or the like.

### Cell Free Colonic Model

This model is similar to the Human Colonic Model described above without the representative colonic cell line (such as CACO-2 or T84).

### Bacteria Free Colonic Model

This model is similar to the Human Colonic Model described above. In this model, the biological modelling device is maintained in sterile environment and not inoculated with bacteria.

### Airway Mode

A representative airway cell line (such as Calu-3, or primary airway culture) is grown in a monolayer on the permeable support of a transwell insert 4. A cell compatible mucus layer is placed on top of cell layer so as to be representative of airway mucin in the model. Bacteria may be optionally introduced in specific examples of the model. The headspace of the apical chambre will be airspace containing gases. A nutrient media is provided in the basolateral chamber.

In this model, as a lung function model, both chambers will be aerobic gaseous environments. The gaseous environments in the two chambers can be modulated to mimic conditions such as: hyper/hypocapnia, hyper/hypoxemia, cigarette smoke, pollution, carbon monoxide poisoning or the like.

### Mucus Free Airway Model

This model is similar to the airway model described above. In this model, the mucus layer is absent.

It will be appreciated by persons skilled in the art that the above detailed examples have been described by way of example only and not in any limitative sense, and that various alterations and modifications are possible without departing from the scope of the invention as defined by the appended claims. Various modifications to the detailed examples described above are possible.

Through the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract or drawings), or to any novel one, or any novel combination, of the steps of any method or process so disclosed.

It will be appreciated by persons skilled in the art that the above embodiment(s) have been described by way of example only and not in any limitative sense, and that various alterations and modifications are possible without departing from the scope of the invention as defined by the appended claims. Various modifications to the detailed designs as described above are possible.

### LIST OF COMPONENTS AND REFERENCE NUMERALS

2 Biological modelling device 43 Pierceable film (second)
4 Chamber (insert) 44 Duct (first)
5 Insert arms 46 Duct (second)
6 Insert bottom surface 48 Pierceable film substrate
8 Insert inner wall 50 Sheet
10 Insert headspace 52 Sheet aperture (third)
12 Chamber (well) 54 Sheet aperture (fourth)
14 Well bottom surface 56 Protrusion
16 Well inner wall 58 Clip
18 Well headspace 60 Passage
20 Porous membrane 62 Recess
22 Seal 64 Transfer cover
23 Gasket
24 Base plate
25 Air valve
26 Skirt
27 Tray
28 Flange
30 Barrier layer
32 Cover
34 Cover flange
36 Latch
38 Cover seal
40 Cover aperture (first)
41 Pierceable film (first)
42 Cover aperture (second)

1. A biological modelling device comprising:
a first chamber providing a first reservoir having a first gaseous environment, a second chamber providing a second reservoir having a second gaseous environment, wherein the first reservoir and second reservoir are connected via a permeable support and the first chamber is at least partially received in the second chamber; and
a seal arranged and sealingly engaging the first and second chambers such that a first gaseous environment of the first reservoir and a second gaseous environment of the second reservoir are separated by the seal.

2. The biological modelling device according to claim 1, wherein the permeable support is a membrane, optionally a gas permeable membrane.

3. The biological modelling device according to claim 1, wherein the first chamber is configured to receive and hold a first biological element and/or a solution and the second chamber is configured to receive and hold a second biological element and/or a solution.

4. The biological modelling device according to claim 1, wherein the first gaseous environment comprises a headspace of the first reservoir of the first chamber, and the second gaseous environment comprises a headspace of the second reservoir of the second chamber.

5. The biological modelling device according to claim 1, wherein the second gaseous environment is sealed from the first gaseous environment by the seal and the first chamber in combination.

6. The biological modelling device according to claim 1, wherein the seal is a gasket seal.

7. The biological modelling device according to claim 6 wherein the gasket seal comprises a pierceable septum, optionally a self-sealing septum.

8. The biological modelling device according to claim 6, wherein the first chamber is partially received through the gasket seal into the second chamber.

9. The biological modelling device according to claim 1, wherein the seal comprises a barrier layer arranged to sealingly engage with the second chamber so as to separate the second gaseous environment from the first gaseous environment, the barrier layer comprising an aperture, wherein the first chamber is partially received through the aperture to form a sealing engagement between the barrier layer and the first chamber whereby the second chamber is sealed by the barrier layer and the first chamber.

10. The biological modelling device according to claim 1, wherein the second chamber is located in a base plate, or wherein the second chamber is formed as part of a base plate.

11. The biological modelling device according to claim 1, wherein the second chamber comprises a bottom surface, an inner wall extending upwardly from the bottom surface towards an open top, the bottom surface and inner wall together defining the second reservoir.

12. The biological modelling device according to claim 1, wherein the first chamber is an insert comprising the permeable support.

13. The biological modelling device according to claim 12, wherein the permeable support forms at least a portion of the bottom surface of the first chamber.

14. The biological modelling device according to any claim 1, comprising a plurality of second chambers, each second chamber comprising a second reservoir.

15. The biological modelling device according to claim 14, comprising a plurality of first chambers, each first chamber being an insert corresponding to each of the plurality of second chambers, each insert comprising a first reservoir, and a permeable support located between the respective first and second reservoirs.

16. The biological modelling device according to claim 15, comprising a plurality of seals, each arranged between and sealingly engaging the respective first chamber and second chamber.

17. The biological modelling device according to claim 1, wherein the first chamber is an apical chamber, and optionally wherein the second chamber is basolateral chamber.

18. The biological modelling device according to any one claim 1, wherein the seal is arranged between and sealingly engaging an outer surface of the first chamber and an inner wall of the second chamber.

19. The biological modelling device according to claim 1, further comprising a cover for the first chamber, wherein the headspace of the first chamber is closed by the cover.

20. The biological modelling device according to claim 19, wherein the cover comprises a seal, arranged to sealingly engage the first chamber so as to separate the first gaseous environment from the surrounding ambient environment, and wherein the headspace of the first chamber is sealed by the seal.

21. The biological modelling device according to claim 1, wherein the seal of the cover comprises at least one pierceable portion.

22. The biological modelling device according to claim 21, wherein the seal of the cover comprises at least two pierceable portions, wherein one pierceable portion is configured to allow access to one of the first chamber or the second chamber, and another of said pierceable portions is configured to allow access to the other of said first chamber and the second chamber.

23. The biological modelling device according to claim 21, wherein the pierceable portion comprises a septum, and optionally wherein the septum is a resealable or self-sealing septum.

24. The biological modelling device according to claim 1 being a culture device for growing cells and/or tissue in vitro.

25. A method of co-culturing cells using the biological modelling device according to claim 1.
